(19) United States
(12) Patent Application Publication
(10) Pub. No.: US 2025/0095791 A1
(43) Pub. Date: Mar. 20, 2025

(54) INFORMATION PROCESSING DEVICE, INFORMATION PROCESSING METHOD AND NON-TRANSITORY COMPUTER READABLE MEDIUM

(71) Applicant: ENEOS Corporation, Tokyo (JP)

(72) Inventors: Taku WATANABE, Tokyo (JP); Yoshihiro YAYAMA, Tokyo (JP)

(73) Assignee: ENEOS Corporation, Tokyo (JP)

(21) Appl. No.: 18/965,509

(22) Filed: Dec. 2, 2024

Related U.S. Application Data
(63) Continuation of application No. PCT/JP2023/019549, filed on May 25, 2023.

(30) Foreign Application Priority Data
May 31, 2022 (JP) 2022-089171

(51) Int. Cl. G16C 20/10; G16C 20/50; G16C 20/70

(52) U.S. Cl. CPC G16C 20/10; G16C 20/50; G16C 20/70

(57) ABSTRACT

An information processing device includes a memory and a processor. The memory stores information on a trained model that outputs a physical property value when information on a molecule is input. The processor defines a molecular model representing a target molecular structure and an adsorbent model representing a structure of an adsorbent, performs a simulation in which the trained model is used at least in part in a first model in which the molecular model is placed around the adsorbent model under arbitrary activity and temperature conditions, and acquires an adsorption volume and adsorption structure as a result of the simulation.

### CROSS REFERENCE TO RELATED APPLICATIONS

This application is continuation application of International Application No JP2023/019549, filed on May 25, 2023, which claims priority to Japanese Application No. 2022-089171, filed on May 31, 2022, the entire contents of which are incorporated herein by reference.

### FIELD

This disclosure relates to an information processing device, an information processing method, and a non-transitory computer readable medium.

### BACKGROUND

There are separation methods for gases, liquids, and others: a pressure swing method and a temperature swing method, which utilize differences in adsorption properties. For high-performance adsorbents, an adsorption mechanism at a molecular level is important, and microstructure control is necessary. When considering substances with such microstructures, a molecular simulation is sometimes used for detailed analysis at the molecular level. When the molecular simulation is used to predict an adsorption state on a substance, calculations are performed using a grand canonical ensemble, a statistical method based on a Monte Carlo method. The grand canonical Monte Carlo (GCMC) method is a well-known Monte Carlo method.

In achieving the GCMC method, energy calculations of adsorbents and adsorbed molecules are required under arbitrary pressure and temperature conditions. Generally, a quantum chemical calculation based on classical theory, including force field and density functional theory is used. However, these methods have problems in satisfying both calculation efficiency and accuracy, and in many cases, they can only be applied to specific targets.

The method using the quantum chemical calculation, which can provide highly accurate predictions, requires advanced modeling because direct calculations are not realistic due to their high calculation load. On the other hand, the method using a classical force field calculation allows a faster and larger-scale calculation than the quantum chemical calculation, but treatment of chemical reactions is limited and accuracy remains an issue. In both methods, the calculation must be performed in a foolproof manner and requires very long time and high calculation costs.

### BRIEF DESCRIPTION OF THE DRAWINGS

FIG. 1 shows a flowchart illustrating an example of processes of an embodiment.

FIG. 2 shows a diagram illustrating an example of a model of an embodiment.

FIG. 3 shows a diagram illustrating an example of an isothermal adsorption curve of an embodiment.

FIGS. 4 and 5 show flowcharts illustrating an example of processes of an embodiment.

FIGS. 6 and 7 show diagrams illustrating an example of a model of an embodiment.

FIG. 8 shows a diagram illustrating an example of implementation of an information processing device of the embodiment.

### DETAILED DESCRIPTION

According to an embodiment, an information processing device includes a memory and a processor. The memory stores information on a trained model that outputs a physical property value when information on a molecule is input. The processor defines a molecular model representing a target molecular structure and an adsorbent model representing a structure of an adsorbent, performs a simulation in which the trained model is used at least in part in a first model in which the molecular model is placed around the adsorbent model under arbitrary activity and temperature conditions, and acquires an adsorption volume and adsorption structure as a result of the simulation.

An embodiment of the present invention will be hereinafter described with reference to the drawings. The drawings and the description of the embodiment are presented by way of example only and are not intended to limit the present invention. In the description, words “or less” and “or more” may be read as “less than” and “greater than,” respectively, and vice versa, as appropriate, to the extent consistent.

A grand canonical ensemble is an ensemble that takes into account an increase and decrease of molecules in an equilibrium state in statistical mechanics. A grand canonical Monte Carlo (GCMC) method is a Monte Carlo method to enable this ensemble.

Under any partial pressure (pᵢ) and temperature (T) of a target gas molecule “i”, a chemical potential μᵢ of an adsorbed molecule in a gas phase is given by the following since an adsorbent is in a thermal equilibrium state with the gas phase.

$${μ}_{i} ( {p}_{i} , T ) = {μ}_{i} ( {p}_{ref} , 0 ) + {μ}_{i} ( {p}_{ref} , T ) + {\tilde{μ}}_{i} ( {p}_{i} , T ) \qquad ( 1 )$$

Where μᵢ(p_{ref}, 0 [K]) is a reference for the chemical potential and represents a total energy given by a molecular simulation, μᵢ(p_{ref}, T) is a temperature dependence of a standard state and is a value acquired from a thermodynamic database such as a handbook, and μᵢ˜ (pᵢ, T) is a pressure dependence of any state and is given by k_{B}T In(pᵢ/p_{ref}) in a case of ideal gas, for example. Processes of fluid input and output are neglected. A liquid phase can also be treated in the same way, but fugacity (fᵢ) must be used instead of partial pressure pᵢ.

In the GCMC method, the chemical potential is calculated based on Equation (1) above, and a process of determining whether transition of the chemical potential is adopted or not is repeated.

### First Embodiment

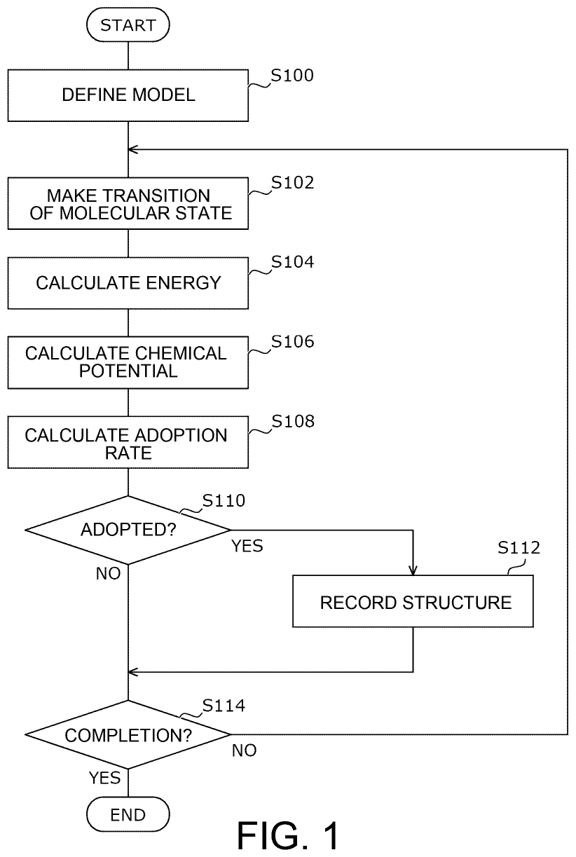

FIG. 1 is a flowchart illustrating processes of the GCMC method. Since this is a generally known algorithm, detailed explanations may be omitted except for characteristic parts in this disclosure. Although energy and potential in an adsorbent and adsorbed molecules shall be used, when other environments are defined, a GCMC simulation can be achieved in the same way by replacing them with appropriate crystalline, amorphous, molecular aggregation, or other structures and molecules, respectively.

Although a case in which GCMC is used as a simulation is described in this embodiment, it is not limited thereto, and NNP may be used in the same way in a simulation that can appropriately optimize a state between an adsorbent and adsorbed molecules. As an example, the energy and chemical potential of a molecule are calculated using NNP, but it is not limited thereto, and other physical property values that can appropriately express the state between the adsorbent and adsorbed molecules may be acquired using NNP.

The simulation is performed by one or more processing circuits (processors) based on appropriate data and programs stored in one or more storage circuits (including memory and storage) in one or more computers, for example. In other words, information processing by software may be concretely achieved using hardware resources such as processing circuits.

For example, an information processing device includes a processing circuit and a storage circuit. The processing circuit executes information processing by software by reading from the storage circuit programs and parameters regarding a trained model that are necessary for the processes illustrated in FIG. 1. As an example, the storage circuit stores information on a trained model that outputs physical property values when molecular information is input, and the processing circuit can appropriately acquire the physical property values from the molecular information by acquiring the parameters of this trained model.

In the simulation, the processing circuit of the information processing device first defines a model (S100). This model is defined with a molecular model representing a target molecular structure (adsorbed molecule) and an adsorbent model representing an adsorbent structure.

The adsorbent has a structure including, for example, at least one of the following: an aggregate of molecules, a liquid, a crystalline or amorphous solid, a cluster, a defect structure, or an interface structure. The adsorbed molecules are molecules of a substance that are adsorbed on the adsorbent. For example, what kind of adsorbent material can efficiently adsorb the target adsorbed molecules is explored by a method and device according to the embodiment described in this disclosure.

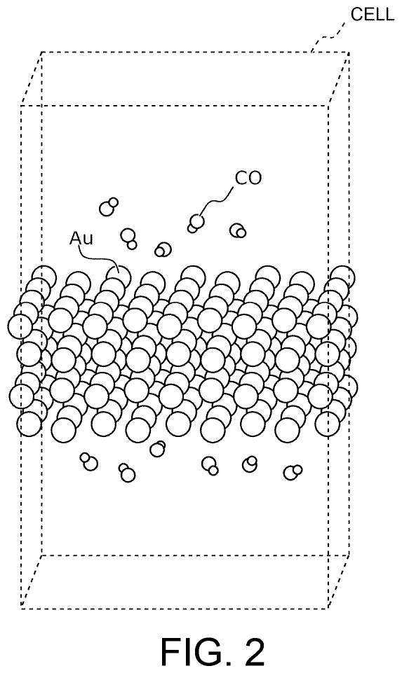

FIG. 2 is a diagram illustrating an example of a model to be defined. As an example, the figure assumes that the target molecule is CO and the adsorbent is Au. This structure is defined, for example, as a periodic boundary condition. In this figure, a layer of Au is provided in a middle of a cell in a vertical direction, but it is not limited thereto. The Au layer may be defined at a bottom or top of the cell, for example, if the cell is appropriately sized.

The processing circuit places Au with a structure having a (111) plane as a surface, as an example. In FIG. 2, Au has the (111) plane as its surface, but it is not limited thereto, and any plane can be used as the surface structure. The processing circuit may also appropriately define the number of Au atoms to a number other than these.

The processing circuit defines the structure of Au as an adsorbent model and forms a molecular model by placing the molecules of CO, the target molecule, around this adsorbent model. The number, direction, and positions of the molecules to be placed can be defined arbitrarily. The processing circuit defines a model in which this adsorbent model and the molecular model are placed as a first model.

Returning to FIG. 1, the processing circuit makes a transition of a state of the molecule (S102) after defining the model. The processing circuit, for example, makes a transition of any molecular model in FIG. 2 within restriction between Monte Carlo steps. This restriction may be, for example, restriction on a distance that can be moved within the time between the Monte Carlo steps, an angle that can be rotated, or other restrictions. The processing circuit makes the transition of the state by adding or desorbing molecules to or from the first model, or by translating or rotating molecules.

The processing circuit calculates energy after the state transition (S104). The processing circuit calculates the energy of the molecule by NNP (neural network potential). The processing circuit builds a trained model by reading parameters of a neural network model according to NNP stored in the storage circuit and calculates the energy of the state in which the molecular model is made the transition from the first model to this trained model.

The processing circuit then calculates the chemical potential after the state transition (S106). Similar to the process in S104, the processing circuit calculates the chemical potential by NNP. The processing circuit builds the trained model by reading the parameters of the neural network model for NNP stored in the storage circuit and calculates the energy of the state in which the molecular model is made the transition from the first model to this trained model. The chemical potential is a quantity specified in Equation (1) and can be calculated by appropriately trained NNP. The trained models used for S104 and S106 may be different models.

A total differential of energy E, with entropy as S, volume as V, and the number of particles as N, is determined as follows.

$$d E = {( \frac{\partial E}{\partial S} )}_{V , N} d S + {( \frac{\partial E}{\partial V} )}_{S , N} d V + {( \frac{\partial E}{\partial N} )}_{S , V} d N \qquad ( 2 )$$

Where a coefficient of dN can be defined as the chemical potential μ=(67 E/∂N)_{S, V}. Expressed in this way, the following equation holds.

$$d E = T d S + p d V + μ dN \qquad ( 3 )$$

From Equation (3), a relationship among energy E, temperature T, partial pressure p, and chemical potential μ is defined. The chemical potential μ is then expressed by Equation (1), which depends on the temperature and partial pressure.

Based on the energy and chemical potential acquired by NNP, the processing circuit calculates an adoption rate of whether or not the insertion, desorption, translation, or rotation of the molecules in the Monte Carlo method is acceptable in the step (S108). The processing circuit calculates the adoption rate in each case. Hereinafter, N is the number of adsorbed molecules, V is an adsorbent volume, and s is an arbitrary state of a system.

When the molecule is inserted, the adoption rate is determined as follows.

$${r}_{a , i} ( N \to N + 1 ) = ( \frac{N}{{Λ}^{3} ( N + 1 )} ) exp [ β \{ {μ}_{i} ( {p}_{i} , T ) - Δ E ( N \to N + 1 ) \} ] \qquad ( 4 )$$

Where i is the identifier of the molecule contained in the first model.

When a molecule is desorbed, the adoption rate is determined as follows.

$${r}_{a , i} ( N \to N - 1 ) = ( \frac{{Λ}_{i}^{3} N}{V} ) exp [ - β \{ {μ}_{i} ( {p}_{i} , T ) ∓ Δ E ( N \to N - 1 ) \} ] \qquad ( 5 )$$

When the molecular state changes (s→s′) by translation or rotation, the adoption rate is determined as follows.

$${r}_{a , i} ( s \to {s}^{′} ) = min [ 1 , exp \{ - βΔ {E}_{i} ( s \to {s}^{′} ) \} ] \qquad ( 6 )$$

Here, in the above, β and Λ are as defined below.

$$β = {k}_{B} T \qquad ( 7 )$$

$${Λ}_{i} = \frac{h}{\sqrt{2 π {m}_{i} {k}_{B} T}} \qquad ( 8 )$$

Where k_{B} is Boltzmann's constant and mi is the mass of the i-th molecule.

The processing circuit selects and calculates the adoption rate of one of Equations (4) to (6) corresponding to insertion, desorption, or translation or rotation, as appropriate, based on the transition state in S102 for each molecule.

The processing circuit determines whether a transition state of the molecule in question is adopted or not based on the calculated adoption rate (S110). For example, the processing circuit acquires a standardized random number, and when this standardized random number is smaller than the adoption rate, the processing circuit adopts the transition state in S102 for that molecule (S110: YES), and when it is the adoption rate or more, the processing circuit determines not to adopt the transition state (S110: NO).

When the state transition of the molecule is adopted (S110: YES), the processing circuit records the structure of the molecule as a processing state in the next Monte Carlo step (S112). The processing circuit may update the structure of the molecule stored in the storage circuit in the current step to this structure of the molecule.

Not limited to the above, the structure of the molecule in the current step may remain stored, for example, in a case when the energy or chemical potential is increasing, rather than updating the structure. In this case, the processing circuit may record the adopted state transition and use it in the next Monte Carlo step. In this way, it may be in the form of recording local solutions of the energy and chemical potential, and a final structure to be used can be selected.

As another example, when the energy and chemical potential are decreasing, the state transition may be adopted without calculating the adoption rate.

After the process of S112 or after no adoption (S110: NO), the processing circuit determines whether or not a simulation completion condition has been met (S114). When the processing circuit determines that the completion condition of the simulation has been met (S114: YES), it completes the process. When the processing circuit determines that the completion condition of the simulation has not been met (S114: NO), it repeats the process from S102.

The completion condition of the simulation can be set arbitrarily. For example, the completion condition may be that the Monte Carlo step has been repeated a predetermined number of times. For example, the completion condition may be that the adoption rate becomes less than a predetermined value. For example, a predetermined condition may be that the energy value or chemical potential value, or a difference between steps of the energy value or a difference between steps of the chemical potential value, is less than a predetermined value.

The processing circuit updates the first model to a model containing the new adsorbent and molecular structure adopted in S110 and recorded in S112 and executes the process from S102.

In at least one of the processes in S104 and S106, the energy or chemical potential value in the previous Monte Carlo step may be required when determining the difference. In such cases, at a subsequent step after the energy and chemical potential are calculated, the processing circuit may store physical property values required in the next Monte Carlo step in the storage circuit as appropriate.

The above simulation may be performed, for example, at a predetermined activity and temperature condition. After performing the simulation under fixed activity and fixed temperature states, the processing circuit makes a transition of the activity or temperature and performs further simulations. In this way, the processing circuit performs the simulation with arbitrary activity and an arbitrary temperature condition using NNP.

The processing circuit executes estimation of the adsorbent structure and adsorption volume for adsorbed molecules based on simulation results acquired at various activities and various temperatures. For this execution, the processing circuit performs the simulations with different adsorbents by changing the above activity and temperature conditions one after another and comprehensively determines the results acquired from the simulation results to acquire an appropriate adsorbent and the adsorption volume of the adsorbed molecules.

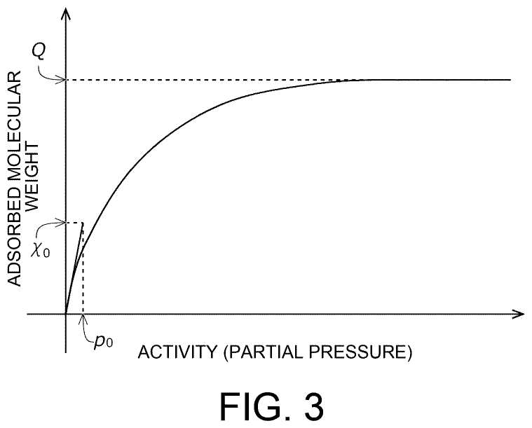

FIG. 3 is a diagram illustrating an example of an isothermal adsorption curve, which is a relationship of an adsorbed molecular weight acquired in the simulation to the activity under isothermal conditions. For example, the processing circuit acquires the adsorbed molecular weight under isothermal and isoactivity conditions for the processes illustrated in FIG. 1. After the acquisition, the processing circuit changes the conditions of the activity without changing the temperature to acquire the adsorbed molecular weight. In this way, the isothermal adsorption curve can be acquired by acquiring the adsorbed molecular weight when the activity is changed under isothermal conditions.

Here, the slope χ₀/p₀ at the origin illustrated in FIG. 3 represents Henry's constant. In addition, molecular weight Q, in which the adsorbed molecular weight is saturated regardless of the activity, represents a maximum adsorption volume. In other words, by acquiring the isothermal adsorption curve, the processing circuit can estimate the Henry's constant and the maximum adsorption volume.

The processing circuit can, for example, estimate the Henry's constant by acquiring the slope by linear regression of the activity and adsorption volume in a low-activity region in the isothermal adsorption curve. The processing circuit can also estimate the maximum adsorption volume, for example, by acquiring the saturated adsorption volume in a high-activity region in the isothermal adsorption curve.

As described above, according to one application of the information processing device and information processing method of this embodiment, it is possible to estimate the Henry's constant and the adsorbed molecular weight for the adsorbent with respect to the adsorbed molecules. This estimation can be achieved at high speed and with the same level of high accuracy as quantum chemical calculations by using NNP for simulations for respective conditions. As a result, the information processing device can handle both physical adsorption and chemical adsorption in parallel. In other words, according to this embodiment, the information processing device can acquire what kind of adsorbent is suitable for a certain adsorbed molecule at a low cost.

In addition, according to the above information processing device, it is possible to achieve highly versatile physical property estimation by using appropriate NNP, since NNP needs only be prepared as a force field.

A database of a relationship between interatomic distance and energy may be created in advance for two or more element types and combinations. By using this database, the processing circuit will be able to achieve GCMC simulations faster and more accurately. This database may be generated using NNP. In particular, speed and accuracy can be improved in cases where structural changes occur in adsorbed molecules or adsorbents, as described below.

### Second Embodiment

In the first embodiment described above, it is not expressed that the adsorbed molecule and adsorbent change structure explicitly, but the method in this disclosure can be used even when the structure of either or both the adsorbed molecule and adsorbent change.

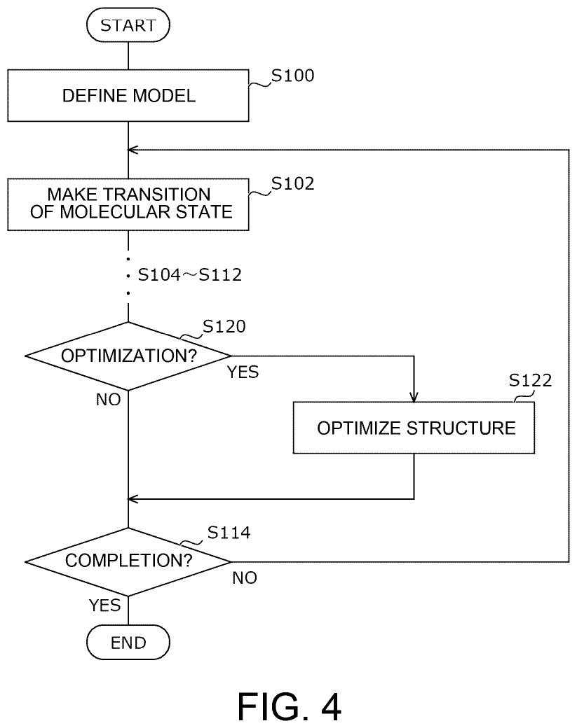

FIG. 4 is a flowchart illustrating an example of processes of the information processing device of this embodiment. The processes indicated by the same codes as in FIG. 1 are the same as in the first embodiment described above. S104 to S112 are omitted from the illustration in the figure.

The processing circuit of the information processing device may perform optimization of the adsorbed molecules and the structures of adsorbents at any given time.

For example, after the process in S110 or S112 is completed, the processing circuit determines whether or not to perform the optimization (S120). This determination condition can be decided arbitrarily. For example, the determination condition for the optimization can be set to a condition such as completion of the calculation of a predetermined number of Monte Carlo steps. As another example, the processing circuit may perform the determination of S120 using the condition that a distance between a certain adsorbed molecule and the adsorbent is closer than a predetermined distance.

When the determination is made not to perform the optimization (S120: NO), the processing circuit continues the same process as in FIG. 1.

When the determination is made to perform the optimization (S120: YES), the processing circuit performs structure optimization (S122). The processing circuit may define the model for which the structure optimization is performed as a new model (second model).

When the simulation is to continue (S114: NO), the processing circuit repeats the processes from S102, using the acquired second model as an initial value for the first model in the next Monte Carlo step.

The process in S122 may be performed using NNP. By using NNP in the structure optimization, it is possible to calculate structural changes in the first model, such as molecular cleavage and structural changes in the adsorbent, which are difficult to deal with in classical theory, to generate the second model.

As described above, this embodiment makes it possible to incorporate molecular cleavage and structural changes in the adsorbent itself into the simulation, which are difficult to deal with in classical theory. As a result, more versatile GCMC simulations can be enabled. In other words, the processing circuit can check a bonding state in the adsorbed molecule during the simulation based on the adsorbent model and perform equilibrium state calculations with the product after the reaction, even in the case of structural changes involving state changes such as bonding and cleavage.

### (Modification Example of Second Embodiment)

The process of S122 is not limited to the structure optimization of molecules, crystals, and the like. For example, as in the process of S122, the processing circuit can perform molecular dynamics simulations using NNP. By performing the molecular dynamics simulation, it is possible to generate the second model that takes into account time evolution of the molecules in the first model.

As yet another example, the processing circuit may perform a canonical Monte Carlo simulation as the process of S122. The canonical Monte Carlo simulation is a simulation that uses an ensemble with constant particle number, volume, and temperature, whereas GCMC uses an ensemble with constant chemical potential, volume, and temperature.

The processing circuit can also use NNP in this canonical Monte Carlo simulation. By performing the canonical Monte Carlo simulation, the processing circuit performs a simulation in a state where the number of particles does not change. As a result, it is possible to acquire the equilibrium state in which the number of particles is kept constant as the second model.

As described above, other simulations can be performed at appropriate times instead of the structure optimization.

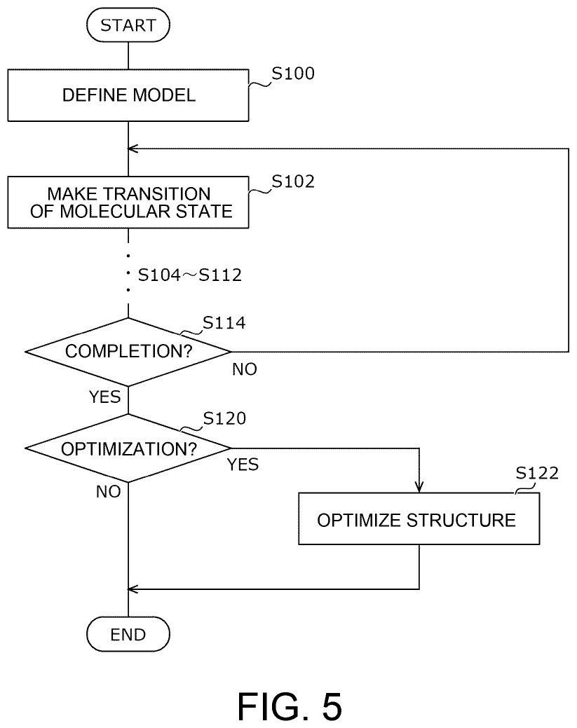

FIG. 5 is an example of a modification of the second embodiment. Instead of performing other types of simulations during GCMC, the processing circuit may perform at least one of the final structure optimization, molecular dynamics simulation, or canonical Monte Carlo simulation using NNP at the completion of GCMC, as illustrated in FIG. 5.

These optimizations and other processes during or at the completion of GCMC may also be used in combination. That is, the processing circuit may perform the structure optimization, molecular dynamics simulation or canonical Monte Carlo simulation, or any combination thereof, during or after the simulation based on the adsorbent model.

More concretely, the processing circuit may be in the form of performing the above optimization and others, or any combination thereof, at either time during or at the completion of GCMC.

For example, the processing circuit may be in the form of performing optimization and others, or any combination thereof during GCMC and not performing the optimization and others, or any combination thereof, at the completion of GCMC.

As another example, the processing circuit may be in the form of not performing the optimization and others, or any combination thereof during GCMC, but performing the optimization and others, or any combination thereof, at the completion of GCMC.

The processing circuit may be in the form of performing the same optimization and others, or any combination thereof, at both times during and at the completion of GCMC.

The processing circuit may also be in the form of performing the optimization and others, or any combination thereof during GCMC, and performing the optimization and others, or any combination thereof at the completion of GCMC that is different from the optimization and others, or any combination thereof, which was performed during GCMC.

Thus, the processing circuit can perform any combination of the above optimization and others, or any combination thereof, at least one of the following times during or at the completion of GCMC.

### Third Embodiment

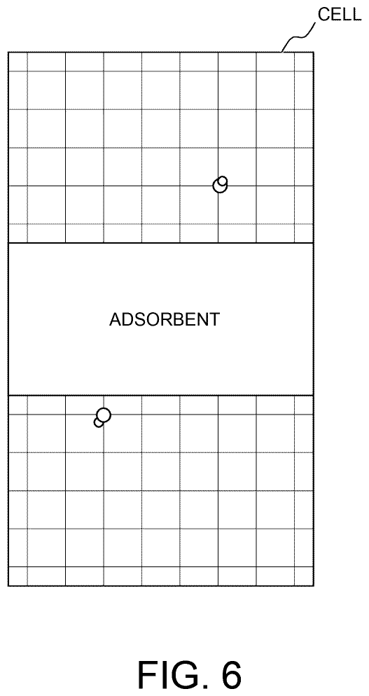

FIG. 6 is a diagram illustrating an example of a model according to a third embodiment. In the following figures, the cell illustrated in FIG. 2 is represented in two dimensions, for example, viewed from the front. Of course, the cell is defined in three dimensions as in FIG. 2.

The processing circuit may have a grid (virtual lattice) within the cell. The processing circuit may provide a restriction that the adsorbed molecules be placed on lattice points of the grid, as an initial first model definition. Also, in FIG. 1, or other figures, when inserting molecules in the process of S102, the adsorbed molecules to be inserted may be placed on the lattice points of the grid.

This lattice point restricts the position at which the molecule is inserted, and the processing circuit does not provide a restriction on displacement of the molecule from the lattice point when the process of S102 generates the state transition such as the desorption, translation, or rotation.

The grid can be set, for example, to avoid beforehand regions where adsorption is impossible within the adsorption structure when determining adsorption equilibrium. By setting such a grid and defining the insertion positions of molecules, it is possible to achieve insertion of adsorbed molecules with the added limitation of avoiding regions where adsorption is impossible. For example, this grid can be set by placement or the like of molecules on a surface of the adsorbent.

The processing circuit may randomly (for example, with equal probability) place molecules for lattice points in the grid when performing at least one of the following: defining the initial first model or inserting the molecules.

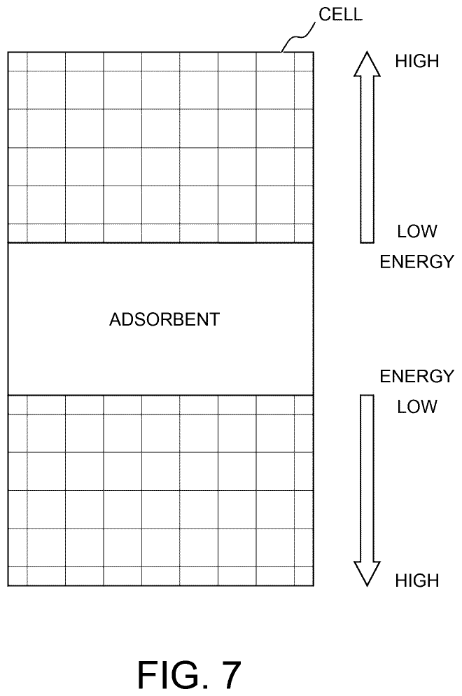

FIG. 7 is a diagram illustrating another placement probability of this embodiment. Arrows on the right of the figure indicate distribution of energy heights of adsorbed molecules when placed in the cell based on their distance from the adsorbent. Each lattice point in the grid is labeled with an energy value in an energy field based on the distance from the adsorbent, as indicated by the arrows on the right.

The processing circuit may perform insertion of the adsorbed molecules based on the labeled energy values. The processing circuit may, for example, control the insertion position of the adsorbed molecules so that the probability of the molecule insertion becomes high as the set energy value becomes low.

In this way, the insertion position of the molecule can be limited and probabilistically defined to a position where it can be easily adsorbed on the adsorbent.

As described above, according to this embodiment, the ease of adsorption can be varied depending on a positional relationship with the adsorbent structure, thereby weighting the adsorbed molecules and improving the adoption rate in simulations where the inserted adsorbed molecules reach an equilibrium state.

Some or all of each device (the information processing device) in the above embodiment may be configured in hardware, or information processing of software (program) executed by, for example, a CPU (Central Processing Unit), or a GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

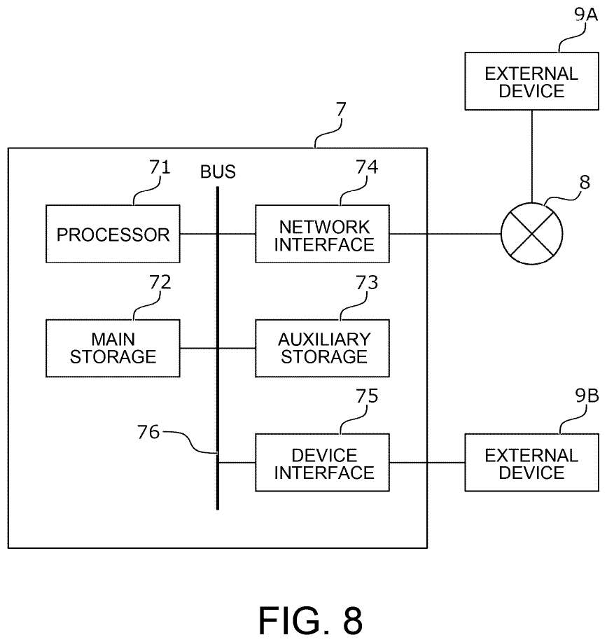

FIG. 8 is a block diagram illustrating an example of a hardware configuration of each device (the information processing device) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 8 is provided with each component one by one but may be provided with a plurality of the same components. Although one computer 7 is illustrated in FIG. 8, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the information processing device) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the information processing device) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73.

In the case of each device (the information processing device) in the above embodiments is configured by at least one storage device (memory) and at least one processor connected/coupled to/with this at least one storage device, the at least processor may be connected to a single storage device. Or the at least storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8.

Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device) in the above embodiments. That is, the computer 7 may transmit a part of or all of processing results to the external device 9A or the external device 9B, or receive a part of or all of processing results from the external device 9A or the external device 9B.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device, comprising:
a memory; and
a processor, wherein
the memory stores information on a trained model that outputs a physical property value when information on a molecule is input,
the processor defines a molecular model representing a target molecular structure and an adsorbent model representing a structure of an adsorbent,
performs a simulation in which the trained model is used at least in part in a first model in which the molecular model is placed around the adsorbent model under arbitrary activity and temperature conditions, and
acquires an adsorption volume and adsorption structure as a result of the simulation.

2. The information processing device according to claim 1, wherein
the trained model is a model regarding NNP (neural network potential), and
the physical property value includes at least energy of the molecule.

3. The information processing device according to claim 2, wherein
the simulation is a grand canonical Monte Carlo simulation.

4. The information processing device according to claim 3, wherein
the processor performs the simulation by creating a database in advance of a relationship between interatomic distances and energies for two or more element types and combinations.

5. The information processing device according to claim 4, wherein
the adsorbent model includes at least one of the following: an aggregation of molecules, a liquid, a crystalline or amorphous solid, a cluster, a defect structure, or an interface structure.

6. The information processing device according to claim 1, wherein
the processor performs the simulation by
acquiring a second model by performing structure optimization, molecular dynamics simulation or canonical Monte Carlo simulation, or any combination of the above during the simulation based on the adsorbent model, and
updating the second model as the first model.

7. The information processing device according to claim 1, wherein
the processor performs the simulation by
acquiring a second model by performing structure optimization, molecular dynamics simulation or canonical Monte Carlo simulation, or any combination of the above after the simulation based on the adsorbent model, and
updating the second model as the first model.

8. The information processing device according to claim 6, wherein
the processor checks a bonding state in the adsorbed molecule during the simulation based on the adsorbent model, and
performs an equilibrium state calculation with a product after a reaction, even when the structure changes with bonding and cleavage.

9. The information processing device according to claim 1, wherein
the processor estimates an isothermal adsorption curve acquired by a relationship between activity and an adsorption volume in the simulation.

10. The information processing device according to claim 9, wherein
the processor estimates Henry's constant from a slope acquired by linear regression of activity and an adsorption volume in a low-activity region in the simulation.

11. The information processing device according to claim 9, wherein
the processor estimates a maximum adsorption volume acquired from a saturated adsorption volume in a high-activity region in the simulation.

12. The information processing device according to claim 1, wherein
the processor performs the simulation by limiting insertion positions of adsorbed molecules in the first model using a virtual lattice.

13. The information processing device according to claim 12, wherein
the processor performs the simulation by
labeling lattice points of the virtual lattice with values of an energy field due to the adsorbent in the first model, and
probabilistically deciding insertion positions of the adsorbed molecules based on the label.

14. The information processing device according to claim 13, wherein
the processor performs the simulation
while increasing a probability of insertion of the adsorbed molecules as an energy value is smaller in the energy field due to the adsorbent in the first model.

15. An information processing method comprising, by a processor:
reading information on a trained model that outputs a physical property value when information on a molecule stored in a memory is input;
defining a molecular model representing a target molecular structure and an adsorbent model representing a structure of an adsorbent;
performing a simulation that uses the trained model at least in part in a first model in which the molecular model is placed around the adsorbent model under arbitrary activity and temperature conditions; and
acquiring an adsorption volume and adsorption structure as a result of the simulation.

16. A non-transitory computer readable medium storing a program causing a processor to execute an information processing method, the information processing method comprising:
reading information on a trained model that outputs a physical property value when input of information on a molecule stored in a memory is input;
defining a molecular model representing a target molecular structure and an adsorbent model representing a structure of an adsorbent; and
performing a simulation that uses the trained model at least in part in a first model in which the molecular model is placed around the adsorbent model, under arbitrary activity and temperature conditions, and
acquiring the adsorption volume and adsorption structure as a result of the simulation.
